(19) United States
(12) Patent Application Publication
(10) Pub. No.: US 2003/0018360 A1
(43) Pub. Date: Jan. 23, 2003

(54) Electric field stimulation (EFS) for high throughput screening

(76) Inventors: Janet K. Robertson, Easton, PA (US); Paul Bennett JR., Doylestown, PA (US)

Correspondence Address: HARNESS, DICKEY & PIERCE, P.L.C., P.O. BOX 828, BLOOMFIELD HILLS, MI, 48303, US

(21) Appl. No.: 10/178,945

(22) Filed: Jun. 24, 2002

Related U.S. Application Data
(60) Provisional application No. 60/299,913, filed on Jun. 22, 2001.

(51) Int. Cl. A61N 1/00

(52) U.S. Cl. 607/2

(57) ABSTRACT

An electric field stimulation (EFS) device is provided for stimulating a plurality of cultured cells. The EFS device includes a transparent substrate, an insulator plate secured adjacent to the transparent substrate and having at least one well formed therethrough for containing the plurality of cultured cells, a surface of the transparent substrate defining a floor of the well, a first transparent electrode disposed on the surface of the transparent substrate for covering at least a portion of the floor, and a second electrode in electrical communication with the first transparent electrode. A voltage is selectively induced across the first transparent electrode and the second electrode for stimulating the plurality of cultured cells.

### CROSS-REFERENCE TO RELATED APPLICATIONS

This application claims the benefit of U.S. Provisional Application No. 60/299,913, filed Jun. 22, 2001.

### FIELD OF THE INVENTION

The present invention relates to screening candidate compounds and more particularly to electric field stimulation of cells for high throughput screening of candidate compounds.

### BACKGROUND OF THE INVENTION

Organic cells include cell membranes which isolate the cell interior from the surrounding environment. These membranes include channels, through which a cell is able to communicate with its environment. These channels are made up of single molecules, or complexes of molecules, and selectively enable the passage of ions therethrough. Over 50% of the pharmaceuticals produced today affect the opening and closing of the ion channels. Therefore, it is important for pharmaceutical researchers to be able to observe the affect of pharmaceuticals on the ion channels.

Ion channels respond to a variety of stimuli including an applied bias across the cellular membrane. Therefore, to understand the operation of ion channels researchers often stimulate cells with an applied voltage. To achieve this, earlier devices included metal micro-electrodes patterned on a glass substrate to stimulate cells and record the subsequent electrical response. These configurations used gold (Au), titanium/platinum (Ti/Pt), or indium tin oxide (ITO) micro-electrodes, that were capped with black platinum (Pt) to reduce electrode impedance and facilitate recording by increasing the signal-to-noise ratio. These devices were used to study the complex signaling between neurons. In particular, these devices were optimized for monitoring one or a few cells, since the primary interest was in understanding the growth, adaptation and signaling between neural networks. These devices, however, were not wholly transparent, and were not optimized for uniform stimulation of multiple cells.

Other devices enable high throughput screening (HTS) of pharmaceuticals and other compounds targeting ion channels. These devices include standard well-plate configurations, which include a modified substrate to electrically stimulate cells. A fluorescent, voltage-sensitive fluid is used to monitor the resulting change in transmembrane potential. These devices are designed to enable light to shine through the substrate, as the optical monitoring equipment of the HTS machines is located below the device.

Three different device configurations for monitoring cell reactions have been previously employed. The first, the dipper electrode configuration, includes two or more electrodes dipped into the top of the wells, and between which a voltage is applied, horizontally across the well. The second is similar to the first, but includes satellite electrodes in an attempt to make the electric field more uniform. Both the first and the second electrode configurations do not modify the bottom surface of the well, and thus, a clear line of sight through the floor of the well is maintained. The third includes electrodes plated within the well, either to the wall of the well, or to a perimeter of the floor of the well. The electrodes in the third configuration are arranged so as to leave a clear line of sight through the floor of the well.

Each of the above described configurations retain specific disadvantages. These disadvantages include (1) requiring cleaning of the electrodes between use, (2) occluding the well openings due to one or more electrodes, and (3) indirectly stimulating the monitored cells, resulting in an inefficient, non-uniform electrical field.

Therefore, it is desirable in the industry to provide an improved HTS device for observing the affect of pharmaceuticals and other compounds on ion channels of organic cells. The device should enable direct, substantially uniform stimulation of the cells, and enable unobstructed optical monitoring thereof. The device should also preferably be configured to enable the passage of light from beneath the device to enable operation with standard HTS machines. Furthermore, the device should minimize the number of electrodes inserted into the well from above.

### SUMMARY OF THE INVENTION

Accordingly, the present invention provides an electric field stimulation (EFS) device for stimulating a plurality of organic cells contained therein. The EFS device includes a transparent substrate, an insulator plate secured adjacent to the transparent substrate and having at least one well formed therethrough for containing the plurality of cultured cells, a surface of the transparent substrate defining a floor of the well, a first transparent electrode disposed on the surface of the transparent substrate for covering at least a portion of the floor, and a second electrode in electrical communication with the first transparent electrode. A voltage is selectively induced across the first transparent electrode and the second electrode for stimulating the plurality of cultured cells.

In accordance with one preferred embodiment, the second electrode is selectively positionable into the top of the well. The EFS device applies a voltage potential vertically, across the cells. In this manner, a substantially uniform voltage is applied, for stimulating the cells. The transparent electrode attached to the floor of the wells enables the passage of light therethrough for improved observation of the cell reactions. In this manner, existing HTS machines retain an unobstructed view of the cells.

In accordance with an alternative preferred embodiment, the second electrode is also transparent and disposed on the surface of the transparent substrate for covering at least a portion of the floor. Preferably, the first transparent electrode and the second electrode include respective inter-digitated fingers. The EFS device applies a voltage potential between the electrodes resulting in electric field lines substantially horizontal to the well floor. In this manner, a substantially uniform voltage is applied, for stimulating the cells. The first and second transparent electrodes attached to the floor of the wells enable the passage of light therethrough for improved observation of the cell reactions.

Further areas of applicability of the present invention will become apparent from the detailed description provided hereinafter. It should be understood that the detailed description and specific examples, while indicating the preferred embodiment of the invention, are intended for purposes of illustration only and are not intended to limit the scope of the invention.

### BRIEF DESCRIPTION OF THE DRAWINGS

The present invention will become more fully understood from the detailed description and the accompanying drawings, wherein:

FIG. 1 is a top view of an EFS device in accordance with the principles of the present invention;

FIG. 2 is a cross-sectional view of the EFS device of FIG. 1, along line 2-2;

FIG. 3 is an expanded, cross-sectional view of a portion of the EFS device, as shown in FIG. 2, detailing operation thereof; and

FIG. 4 is a detailed view of a single well illustrating an alternative electrode configuration.

### DETAILED DESCRIPTION OF THE PREFERRED EMBODIMENTS

The following description of the preferred embodiments is merely exemplary in nature and is in no way intended to limit the invention, its application, or uses.

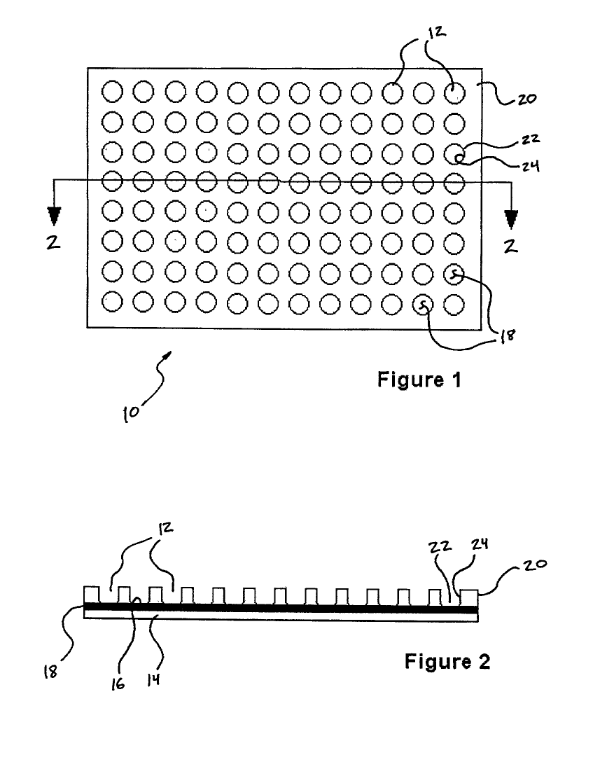

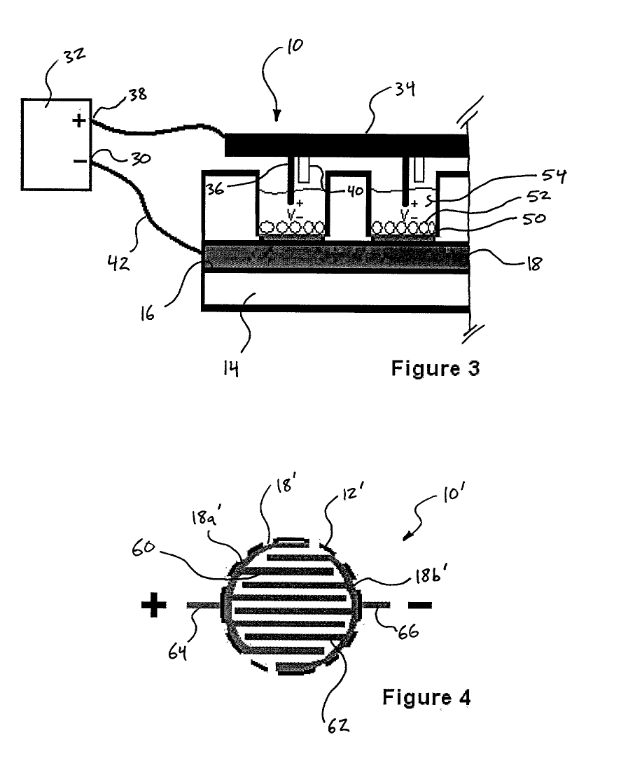

With particular reference to FIG. 1, an electric field stimulation (EFS) device 10 is shown. The EFS device 10 includes a plurality of wells 12 formed therein for retaining organic cells, as described in further detail below. EFS device 10 includes ninety-six (96) wells 12 formed therein with center-to-center spacing and diameters corresponding to industry standards for micro-well plates. Further, per industry standard, the wells 12 are arranged in eight (8) rows by twelve (12) columns. It is anticipated, however, that although the number, size and configuration of the wells 12 is provided per industry standard, each may be varied in accordance with particular design requirements. More specifically, the EFS device can be formed to include 384 or 1536 wells 12. The EFS device 10 is designed for operation with industry standard, high-throughput screening (HTS) machines, such as a fluorometric imaging plate reader (FLIPR®).

FIG. 2 is a cross-sectional view of the EFS device 10 along line 2-2 of FIG. 1. The EFS device 10 includes a substantially transparent substrate 14, preferably manufactured from glass or some other transparent material. The transparent substrate 14 includes a surface 16, to which a transparent electrode 18 is attached. In one embodiment, the transparent electrode completely covers the surface 16 of the transparent substrate 14. As described in further detail herein, other preferred configurations for the transparent electrode 18 are anticipated. By completely covering the transparent substrate, a common electrode is provided for each of the wells 12, as described in further detail herein.

In accordance with a preferred embodiment, the transparent electrode 18 comprises indium tin oxide (ITO). However, it will appreciated that the transparent electrode 18 may be made from any other electrically conductive, transparent material known in the art. An insulator plate 20, having apertures 22 formed therethrough is then bonded to the transparent substrate 14 and transparent electrode 18 combination with a bio-compatible adhesive. In this manner, a circumferential wall 24 of each aperture 22 defines a wall of each well 12, and the surface 16 defines a floor of each well 12. The insulator plate 20 is preferably opaque to prevent light cross-talk between wells 12.

The transparent electrode 18 electrically communicates with a terminal 30 of an external power source 32 that is selectively attachable to the transparent electrode 18. A movable robotic arm 34 includes a series of electrodes 36 extending downward therefrom, which are constructed from an opaque, bio-compatible material. The number of electrodes 36 generally corresponds with the number of wells 12 in a row or the number of wells 12 in a column. The electrodes 36 electrically communicate with another terminal 38 of the external power source 32, and are dipped into the wells 12 for selective inducement of a voltage potential within the wells 12. More specifically, the voltage potential is selectively induced across the electrode 36 and the transparent electrode 18, vertically within each well 12. In this manner, a substantially uniform voltage is applied through the well 12 for more effective stimulation of the cells. It will be appreciated that, although transparent electrode 18 is shown connected to the negative terminal of the power supply 32, and electrode 36 is shown connected to the positive terminal of the power supply 32, the opposite polarity connection can be employed. Further, both AC and DC voltages can be applied.

The robotic arm 34 is a modified version of a standard HTS robotic arm. In particular, standard robotic arms include fluid dispensing conduits for simultaneously dispensing liquid into each well of a column or a row. However, the robotic arm of the present invention includes electrodes 36 for each well 12 of a row (i.e. 8 electrodes), as well as, one fluid dispensing conduit 40 for each well 12 of a row (i.e. 8 fluid dispensing conduits). In this manner, the robotic arm 34 is able to dispense fluid into each of the wells 12 and induce the voltage potential in each well 12, along a particular row.

As described above, the transparent electrode 18 electrically communicates with the power source 32 via a lead 42. Alternatively, however, the transparent electrode 18 may electrically communicate with the power source 32 through an electrode 36. More particularly, the eighth electrode 36 in the row may electrically communicate with the negative terminal 30 of the power source 32, and be longer than the other electrodes 36. As the robotic arm 34 dips the electrodes 36 into the wells 12, the eighth electrode 36 contacts the floor (i.e. transparent electrode 18) of the eighth well 12, to establish an electrical connection. In such a case, there are no cells cultured in the eighth well 12. In this manner, a separate lead from the EFS device is not required, thereby simplifying the EFS device and usage thereof.

In operation, a growth enhancing coating 50, of a type known in the art, is preferably applied to the floor of the wells 12. It will be appreciated, however, that the coating 50 may not be required. Organic cells 52 are selectively placed within the wells 12, using a variety of methods known in the art. These cells 52 may be cultured within the wells 12 for a predetermined period of time. After the cells 52 have cultured for a predetermined period of time, a voltage sensitive, fluorescent fluid 54 is dispensed from the fluid dispensers 40 into the wells 12. The fluid 54 interacts with the cells 52 for facilitating observation.

A voltage potential is applied across the electrode 36 and the transparent electrode 18 for stimulating the cells 52. In response to the applied voltage, a transmembrane potential of the cell changes. The fluorescent fluid 54 reacts to this change, itself changing fluorescence. In this manner, cellular reaction to the applied voltage can be observed. In particular, the effect of pharmaceuticals on the cell membrane ion channels can be determined. Pharmaceuticals, or other compounds may be added to observe their effect on the cells 52.

With particular reference to FIG. 4, an alternative preferred embodiment of the EFS device, indicated as 10′, will be described in detail. The EFS device 10′ includes a patterned, transparent electrode 18′. The transparent electrode covers a portion of the floor of a well 12′ (shown in phantom). More particularly, the transparent electrode 18′ includes a negative electrode portion 18a′ and a positive electrode portion 18b′ having inter-digitated fingers 60, 62, respectively. The electrode portions 18a′,18b′ include respective leads 64,66 for interconnection with the power source 32. Although only a single well 12′ is shown including the patterned transparent electrode 18′, it is anticipated that all of the wells 12 may include such a patterned, transparent electrode, interconnected to form a circuit.

In operation, the configuration of FIG. 4, removes the need for an external electrode to be dipped into the well 12′. Instead, the electric field is induced between the inter-digitated fingers 60, 62, horizontally across the floor of the well 12′. The inter-digitated fingers 60, 62 are formed of such a width and have such a spacing between the electrodes so that a cell 52 typical contacts two or more electrodes 18a′,18b′ (one positive and one negative). This results in very efficient stimulation of the cells. Different widths of electrodes 18a′,18b′ and spacing therebetween can be implemented for varying cell types having different average cell diameters.

The EFS device 10 applies a voltage potential vertically across the cells 52. In this manner, cells 52 in the cell layer are substantially uniformly stimulated. Alternatively, the EFS device 10′ applies a voltage between two electrodes on the floor of the well 12, creating an electric field which is substantially horizontal with respect to the floor. In this manner, cells 52 in the cell layer are also substantially uniformly stimulated. In both configurations, the cells 52 are in direct contact with one or more electrode. Further, the transparent electrodes 18, 18′ covering the floor of the wells 12, enables the passage of light therethrough for observation of the cell reactions. Therefore, existing HTS machines retain an unobstructed view of the stimulated cells 52.

The description of the invention is merely exemplary in nature and, thus, variations that do not depart from the gist of the invention are intended to be within the scope of the invention. Such variations are not to be regarded as a departure from the spirit and scope of the invention.

What is claimed is:

1. An electric field stimulation device for stimulating a plurality of organic cells contained therein, comprising:
a substantially transparent substrate;
an insulator plate secured adjacent to the transparent substrate and having at least one well formed therethrough for containing the plurality of cultured cells, a surface of the transparent substrate defining a floor of the well;
a first transparent electrode disposed on the surface of the transparent substrate for covering at least a portion of the floor; and
a second electrode in electrical communication with the transparent electrode to enable selective application of a voltage thereacross for stimulating the plurality of cultured cells.

2. The electric field stimulation device of claim 1, wherein the transparent electrode is comprised of indium tin oxide (ITO).

3. The electric field stimulation device of claim 1, wherein the second electrode is selectively positioned within the well.

4. The electric field stimulation device of claim 3, further comprising a robotic arm having the second electrode extending therefrom, the robotic arm movable for selectively positioning the second electrode within the well.

5. The electric field stimulation device of claim 1, wherein the second electrode is disposed on the surface of the transparent substrate for covering at least a portion of the floor.

6. The electric field stimulation device of claim 5, wherein the first transparent electrode and the second electrode include respective inter-digitated fingers.

7. The electric field stimulation device of claim 5, wherein the second electrode is also transparent.

8. The electric field stimulation device of claim 7, wherein the second electrode is comprised of indium tin oxide (ITO).

9. The electric field stimulation device of claim 1, wherein said insulator plate is opaque.

10. The electric field stimulation device of claim 1, further comprising a plurality of wells formed in the insulator plate, each having a floor defined by the surface of the transparent substrate.

11. The electric field stimulation device of claim 10, wherein said plurality of wells are formed in a series of rows and columns.

12. The electric field stimulation device of claim 11, wherein said plurality of wells includes one of 96 wells, 384 wells, and 1536 wells.

13. The electric field stimulation device of claim 1, wherein the transparent electrode completely covers a surface of the transparent substrate.

14. The electric field stimulation device of claim 1, wherein the transparent electrode communicates with a first terminal of a power source and the second electrode communicates with a second terminal of the power source.

15. The electric field stimulation device of claim 1, wherein the voltage is applied substantially horizontal with respect to the transparent substrate.

16. The electric field stimulation device of claim 1, wherein the voltage is applied substantially vertical with respect to the transparent substrate.

17. The electric field stimulation device of claim 6, wherein the inter-digitated fingers include a width and spacing such that a single cell may contact at least two or more opposite polarity electrodes.

18. An electric field stimulation device for stimulating a plurality of cultured cells contained therein, comprising:
a substantially transparent substrate;
an insulator plate secured adjacent to the transparent substrate and having at least one well formed therethrough for containing the plurality of cultured cells, a surface of the transparent substrate defining a floor of the well;
a first transparent electrode disposed on the surface of the transparent substrate for covering at least a portion of the floor; and
a second transparent electrode disposed on the surface of the transparent substrate for covering at least a portion of the floor to enable selective application of a voltage across the first transparent electrode and the second transparent electrode.

19. The electric field stimulation device of claim 18, wherein said insulator plate is opaque.

20. The electric field stimulation device of claim 18, further comprising a plurality of wells formed in the insulator plate, each well having a floor defined by the surface of the transparent substrate.

21. The electric field stimulation device of claim 18, wherein the first transparent electrode is in communication with a first terminal of a power source and the second transparent electrode is in communication with a second terminal of the power source.

22. The electric field stimulation device of claim 18, wherein the first transparent electrode and the second transparent electrode include respective inter-digitated fingers.

23. The electric field stimulation device of claim 18, wherein the first and second transparent electrodes comprise indium tin oxide (ITO).

24. The electric field stimulation device of claim 18, wherein the voltage is applied substantially horizontal with respect to the transparent substrate.

25. The electric field stimulation device of claim 22, wherein the inter-digitated fingers include a width and spacing such that a single cell may contact at least two or more opposite polarity electrodes.
